(19) United States
(12) Patent Application Publication
(10) Pub. No.: US 2014/0309175 A1
(43) Pub. Date: Oct. 16, 2014

(54) POLYMERIC CONJUGATES OF C1-INHIBITORS

(71) Applicant: Belrose Pharma, Inc., Princeton, NJ (US)

(72) Inventors: Hong Zhao, Edison, NJ (US); Karen Yang, Edison, NJ (US)

(21) Appl. No.: 14/358,986

(22) PCT Filed: Mar. 15, 2013
(86) PCT No.: PCT/US2013/032122

§ 371 Date: May 16, 2014

Related U.S. Application Data
(60) Provisional application No. 61/612,213, filed on Mar. 16, 2012.
(60) Provisional application No. 61/749,840, filed on Jan. 7, 2013.
(60) Provisional application No. 61/749,842, filed on Jan. 7, 2013.
(60) Provisional application No. 61/749,848, filed on Jan. 7, 2013.

(51) Int. Cl. C07K 14/47

(52) U.S. Cl. CPC C07K 14/4703
USPC 514/20.3; 530/350; 530/410

(57) ABSTRACT

Polymer conjugates containing a C1-inhibitor having at least one substantially non-antigenic polymer covalently attached to the C1-inhibitor via thiol group of the C1 inhibitor is provided. In the polymer conjugates of the present invention, the substantially non-antigenic polymer is attached to either free thiol from a cysteine of thiol generated from disulfide bonds in C1 inhibitor. Alternatively, the substantially non-antigenic polymer is attached to one of more thiols in C1 inhibitor via bifunctional spacer. In addition, methods of making the conjugates as well as methods of treatment using the conjugate of the present invention are also provided.

### CROSS-REFERENCE TO RELATED APPLICATIONS

This application claims the benefit of priority from U.S. Provisional Patent Application Ser. Nos. 61/612,213 filed Mar. 16, 2012, and 61/749,840, 61/749,842 and 61/749,848 filed Jan. 7, 2013, the contents of each of which are incorporated herein by reference.

### FIELD OF INVENTION

The present invention relates to polymeric conjugates containing a C1-inhibitor having at least one substantially non-antigenic polymer covalently attached to the C1-inhibitor via a thiol group of the C1 inhibitor and uses thereof.

### BACKGROUND OF THE INVENTION

C1-inhibitor is a normal constituent of human plasma and belongs to the group of serine protease inhibitors (serpins). One type of C1-inhibitor, C1 esterase inhibitor, is a soluble, single-chain glycoprotein containing 478 amino acid residues. The plasma concentration of C1-esterase inhibitor in a healthy human body is approximately 270 mg/L.

C1-inhibitor is a down-regulator of inflammatory processes in blood. Unlike most family members, C1-inhibitor has a 2-domain structure: the C-terminal serpin domain, which is similar to other serpins, and the N-terminal domain. Structural analysis showed the N-terminal is highly glycosylated leaving the C-terminal more susceptible to reactive binding sites.

Deficiency of this protein is associated with hereditary angioedema or angioneurotic edema, or swelling due to leakage of fluid from blood vessels into connective tissue. Symptoms include swelling of the face, mouth and/or airway that occurs spontaneously or by minimal triggers (such as mild trauma). Such swelling can also occur in any part of the body. In some cases, the levels of C1-inhibitor are low, while in others the protein circulates in normal amounts but it is dysfunctional. In addition to the episodes of facial swelling and/or abdominal pain, it also can cause more serious or life threatening indications, such as autoimmune diseases or lupus erythematosus.

In people with hereditary angioedema, Cinryze® is used to prevent attacks of angioedema, when the C1-esterase inhibitor does not function properly or occurs in low levels, while Berinert® is used to treat attacks of angioedema. Cinryze® is administered at a dose of 1,000 units intravenously at 1 mL/min for 10 min, every 3 or 4 days for routine prophylaxis against angioedema attacks, and Berinert® is administered at a dose of 20 units per kg body weight intravenously at 4 mL/min. Accordingly, non-compliance is a major obstacle to the effective delivery of the C1-esterase inhibitor.

In spite of previous efforts, there is still an unmet need for an improved form of a C1-inhibitor. For example, it would be beneficial to provide long acting C1-inhibitors so that the frequency of dosing could be reduced. The present invention addresses this need.

### SUMMARY OF THE INVENTION

Accordingly, in order to provide the desired improvement, the present invention provides a polymer conjugate containing a C1-inhibitor having at least one substantially non-antigenic polymer covalently attached to the C1-inhibitor via a thiol group of the C1 inhibitor. In another aspect of the invention, polymer conjugates are provided in which one of the substantially non-antigenic polymers is attached to the cysteine of C1-inhibitor. In another aspect of the invention, polymer conjugates are provided in which one of the substantially non-antigenic polymers is attached to a thiol generated from a disulfide bond on the C1-inhibitor. In the present invention, the polymer is attached to a thiol found on the C1-inhibitors via permanent or releasable spacers.

Methods of making the conjugates as well as methods of treatment using the conjugates of the present invention are also provided. Advantages will be apparent from the following description.

### DETAILED DESCRIPTION OF THE INVENTION

In one aspect of the present invention, the polymer conjugate of a C1-inhibitor having at least one substantially non-antigenic polymer covalently attached thereto via one of more C1 inhibitor thiol groups is provided.

The substantially non-antigenic polymer is preferably a polyalkylene oxide such as a polyethylene glycol.

In yet another embodiment, polymer conjugates are provided where the C1-inhibitor is a human C1 esterase inhibitor (C1-INH) or a polypeptide represented by SEQ ID NO: 1 or SEQ ID NO: 2.

In a further embodiment, polymer conjugates are provided in which a substantially non-antigenic polymer is attached to a thiol of a naturally occurring or recombinantly engineered cysteine found on the C1-inhibitor.

In another aspect of the invention, C1-inhibitor polymer conjugates are provided in which a substantially non-antigenic polymer is attached to a thiol group generated from a disulfide bond found in the C1-inhibitor. The C1-inhibitor is treated under conditions to break the disulfide bond and generate two thiol moieties. The thiols are then either conjugated with an activated polymer to provide the polymer conjugates or further reacted with a small bifunctional moieties or spacers before being conjugated with the activated polymer. Some disulfide bonds in C1-inhibitors are located in structurally hindered regions of the C1-inhibitor and thus, reaction conditions are employed to temporarily change the conformation of C1-inhibitor to expose the disulfide and even facilitate reductive hydrolysis, if needed, followed by reaction with a spacer or an activated polymer.

Conditions employed to change the conformation of C1-inhibitor include, but not limited, contacting the C1 inhibitor with a) high concentrations, e.g. from about 1 M to about 10 M of a salt such as guanidinium, guanidine hydrochloride, EDTA, or urea; b) using sufficient amounts of protein denaturing reagents or conditions such as high or low pH than pH 7.4, heavy metals or increased salinity at concentrations of from about 1 M to about 10 M; c) at temperatures ranging from about 45 C to about 100° C. or combinations thereof.

In a further aspect of the invention, C1-Inhibitor-polymer conjugates are provided having at least one substantially non-antigenic polymer attached to a thiol from a naturally or recombinantly engineered cysteine and another polymer attached to a thiol generated from a disulfide bond of the C1-inhibitor, optionally through a bifunctional spacer.

The polymer conjugates of the invention retain about 20% and preferably about 40-80% of the biological activity of the native (unconjugated) C1-inhibitor.

The C1-Inhibitor-polymer conjugates correspond to formula (I) or (I′):

[PEG-(CH₂)ₙ-(L)ₘ]ₚ—(X)_{p′}—C1-inhibitor (I) or

[PEG-(CH₂)ₙ-(L)ₘ]ₚ—(X)_{p′}—C1-inhibitor-(X′)_{q′}-[(L′)_{m′}-(CH₂)_{n′}-PEG]_{q′} (I′)
wherein
PEG is a linear, branched or multi-arm poly(ethylene glycol) having a terminal group —(CH₂CH₂O)—;
L or L′ is independently a linker or functional group suitable to react with thiol; (m) or (m′) is independently 0 or 1;
(n) or (n′) is independently zero or a positive integer, preferably selected from 1, 2, 3, 4, 5, 6, 7, 8, 9, or 10;
(p) or (q) is independently a positive integer, preferably selected from 1, 2, 3, 4, 5, 6 or 7, and more preferably is less than or equal to the number of available cys residues or disulfide groups on the C1-Inhibitor which are available; and
X or X′ is S, a thiol group of an amino acid or a thiol group generated from a disulfide bond in C1-inhibitor attached to the polymer; (p′) or (q′) is independently a positive integer same as (p) or (q), respectively, provided that (m), (m′), (n) and (n′) are not zero simultaneously.

In one aspect of the invention, in the polymer conjugate of Formula (I) or (I′) described above, (n) or (n′) is a positive integer selected from among 1, 2, 3, 4, 5, 6 or 7 and (p) or (q) is a positive integer selected from among of 1, 2 or 3.

In another embodiment, in the polymer conjugate of Formula (I) or (I′) described above, L or L′ is selected from bifunctional moieties which contains at least one chemically blocked or protected functional group and at least one reactive or an activated functional group which reacts during the first conjugation with polymer or C1 inhibitor. In further aspect, the reactive or activated functional group reacts with a thiol more preferably over other functional groups.

### Polymers

In one preferred embodiment, the polymer conjugate described herein can employ a variety of water soluble polymers which have the following formula:

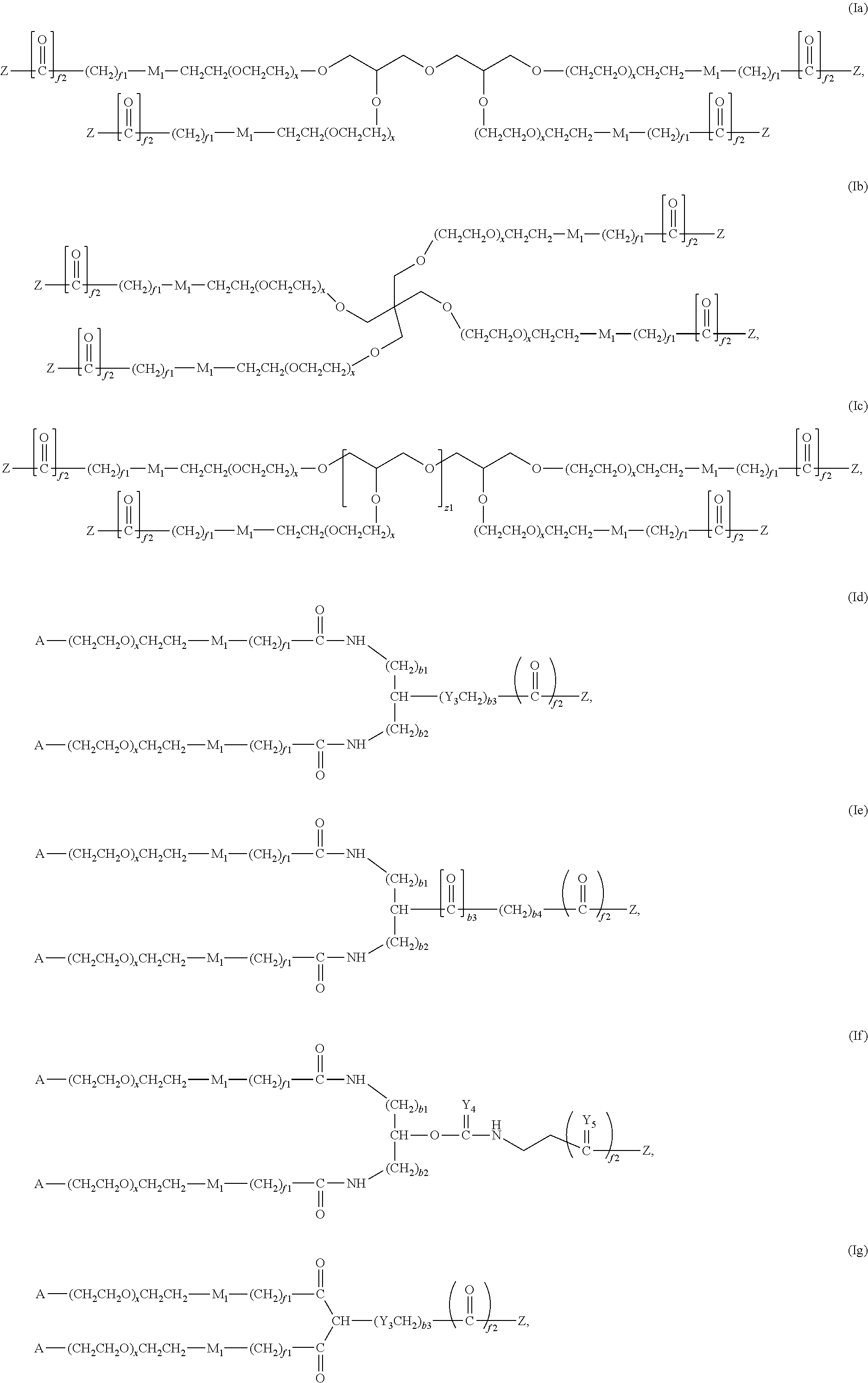

Z—[C(═O)]_{f2}—(CH₂)_{f1}-M₁-CH₂CH₂—O—(CH₂CH₂O)ₓ—CH₂CH₂-M₁-(CH₂)_{f1}-[C(═O)]_{f2}—Z, (Ih)

and

A-(CH₂CH₂O)ₓ—CH₂CH₂-M₁-(CH₂)_{f1}-[C(═O)]_{f2}—Z, (Ii)
wherein
A is hydroxyl, NH₂, CO₂H, or C₁₋₆ alkoxy;
M₁ is O, S, or NH;
Y₃ is O, NR₅₁, S, SO or SO₂;
Y₄ and Y₅ are independently O, S or NR₅₁;
R₅₁, in each occurrence, is independently hydrogen, C₁₋₈ alkyl, C₁₋₈ branched alkyl, C₁₋₈ substituted alkyl, aryl, or aralkyl;

Z, in each occurrence, is independently OH, a leaving group, a targeting group, C₁₋₈ alkyl, C₁₋₈ alkoxy or C1 inhibitor containing moiety;

(b1) and (b2) are independently zero or positive integers;

(b3) is zero or 1;

(b4) is a positive integer;

(f1) is zero or a positive integer of from about 1 to about 10;

(f2) is zero or 1;

(z1) is zero or a positive integer of from 1 to about 27;

(n) is a positive integer of from about 10 to about 2,300 so that the polymeric portion of the conjugate has the total number average molecular weight of from about 2,000 to about 100,000 daltons; and

all other variables are the same as previously defined;

provided that one or more Z is a C1-inhibitor containing moiety.

In a certain embodiment, the molecular weight of the substantially non-antigenic polymer ranges from about 2,000 to about 60,000 daltons, preferably the molecular weight of the substantially non-antigenic polymer ranges from about 5,000 to about 50,000 daltons, and more preferably from about 20,000 to about 40,000 daltons.

In another embodiment, the substantially non-antigenic polymer is conjugated via a linker. In yet another embodiment, the substantially non-antigenic polymer is conjugated via thiol, thioether bond, thiocarbamate or thioamide bond.

For purposes of the present invention, the term “residue” shall be understood to mean that portion of a conjugate, to which it refers, e.g., amino acid, etc. that remains after it has undergone a substitution reaction with another conjugate.

For purposes of the present invention, the term “polymeric containing residue” or “PEG residue” shall each be understood to mean that portion of the polymer or PEG which remains after it has undergone a reaction with C1-inhibitor.

For purposes of the present invention, the term “alkyl” shall be understood to include straight, branched, substituted, e.g. halo-, alkoxy-, nitro-, C₁₋₁₂, but preferably C₁₋₄ alkyls, C₃₋₈ cycloalkyls or substituted cycloalkyls, etc.

For purposes of the present invention, the term “substituted” shall be understood to include adding or replacing one or more atoms contained within a functional group or conjugate with one or more different atoms.

For purposes of the present invention, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

The terms “effective amounts” and “sufficient amounts” for purposes of the present invention shall mean an amount which achieves a desired effect or therapeutic effect as such effect is understood by those of ordinary skill in the art.

According to the present invention, polymers contemplated within the conjugates described herein are preferably water soluble and substantially non-antigenic, and include, for example, polyalkylene oxides (PAO's). The conjugates described herein further include linear, branched, or multi-armed polyalkylene oxides. In one preferred aspect of the invention, the polyalkylene oxide includes polyethylene glycols and polypropylene glycols. More preferably, the polyalkylene oxide includes polyethylene glycol (PEG).

PEG is generally represented by the structure:

—(CH₂CH₂O)ₓ—

where (x) is a positive integer of from about 10 to about 2300 so that the polymeric portion of the conjugates described herein has a number average molecular weight of from about 2,000 to about 100,000 daltons.

The polyalkylene oxide has a total number average molecular weight of from about 2,000 to about 100,000 daltons, preferably from about 5,000 to about 60,000 daltons. The molecular weight of the polyalkylene oxide can be more preferably from about 5,000 to about 25,000 or from about 20,000 to about 45,000 daltons. In some particularly preferred embodiments, the conjugates described herein include the polyalkylene oxide having a total number average molecular weight of from about 30,000 to about 45,000 daltons. In one particular embodiment, a polymeric portion has a total number average molecular weight of about 40,000 daltons.

Alternatively, the polyethylene glycol is further functionalized as represented by the structure:

—[C(═O)]_{f2}—(CH₂)_{f1}-M₁-CH₂CH₂(OCH₂CH₂)ₙ—O-A

wherein

M₁ is O, S, or NH;

(f1) is zero or a positive integer of from about 1 to about 10, preferably, 0, 1, 2, or 3, more preferably, zero or 1;

(f2) is zero or one;

(n) is a positive integer of from about 10 to about 2,300; and

A is hydroxyl, NH₂, CO₂H, or C₁₋₆ alkoxy.

In one embodiment, A is methoxy.

In certain embodiments, all four of the PEG arms can be converted to suitable activating groups, for facilitating attachment to the specific C1-Inhibitor targets, e.g. thiol, etc. or other molecules (e.g., bifunctional linkers). Such conjugates prior to conversion include:

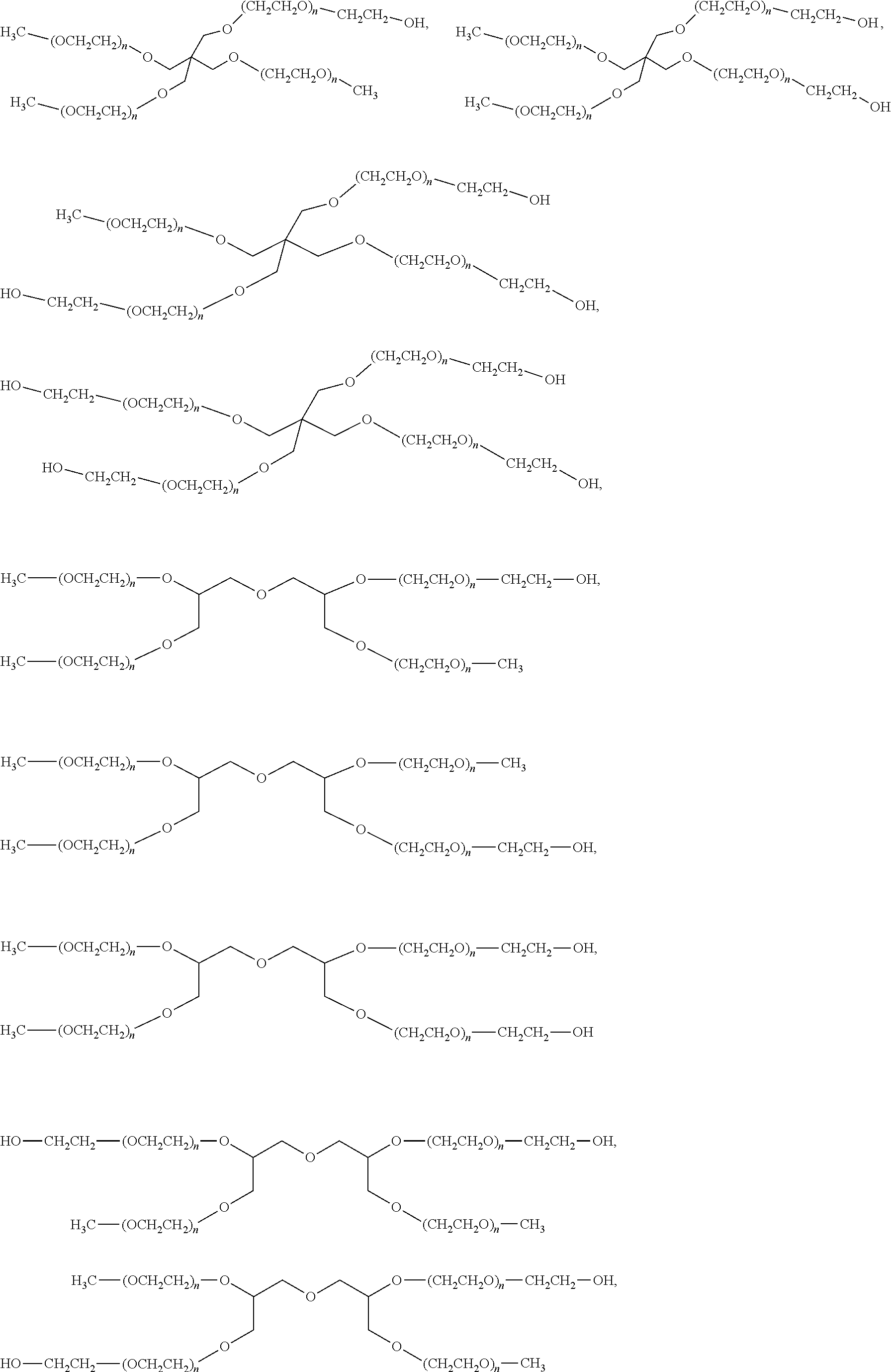

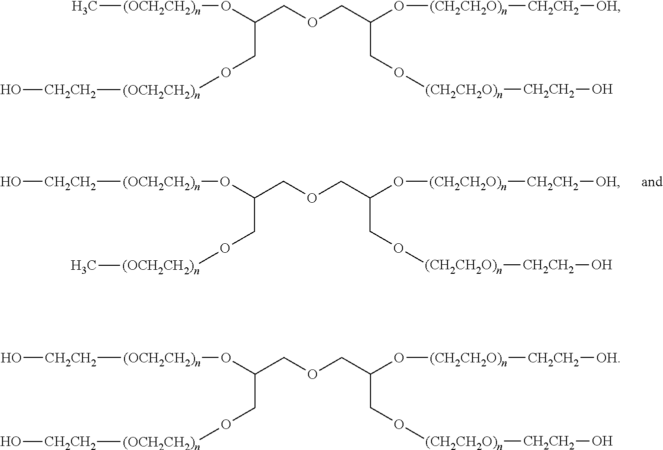

At least one, if not all PEG arms should include maleimide or sulfone other thio pegylating linker.

In yet a further aspect of the invention, the polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In yet a further aspect and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will appreciate that the foregoing list is merely illustrative and that all polymeric materials having the qualities described herein are contemplated. For purposes of the present invention, “substantially or effectively non-antigenic” means polymeric materials understood in the art as being nontoxic and not eliciting an appreciable immunogenic response in mammals.

### Linkers

In one aspect, the substantially non-antigenic polymer of the present invention is conjugated to C1-inhibitor via thioether, thioamide bond or thiocarbamate bond, preferably via thioether.

In one aspect, the substantially non-antigenic polymer of the present invention is conjugated to C1-inhibitor via a linking moieties or a bifunctional spacer.

In some embodiment, the bifunctional moieties contain a residue of a bifunctional spacer such as,

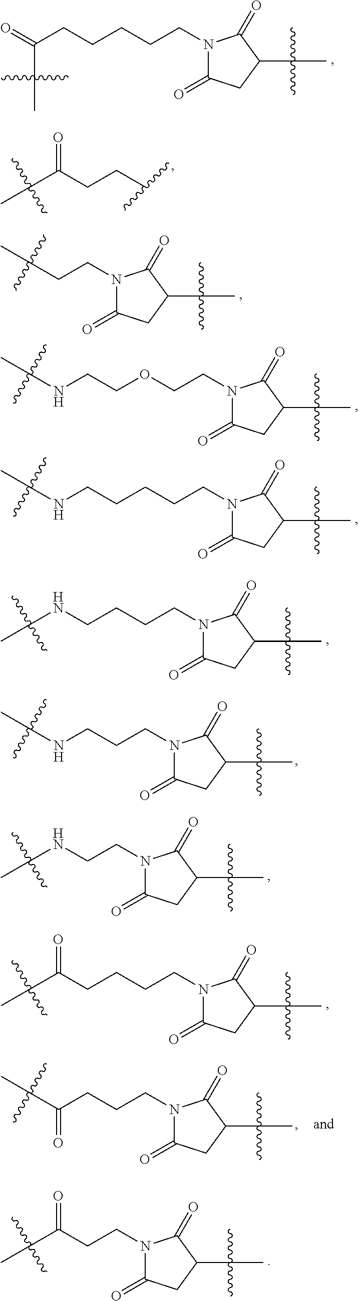

The bifunctional moieties are provided by bifunctional compounds containing a vinyl moiety (—C═C—) such as, but not limited, maleimide or sulfone,

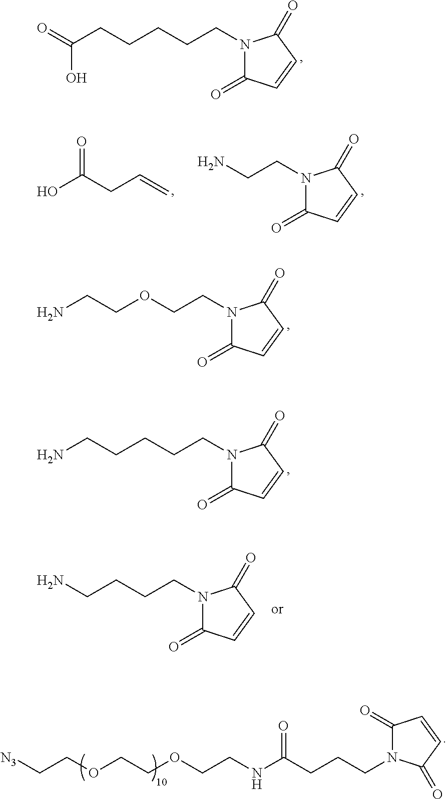

In other aspect, the bifunctional compound contains an activated disulfide bond, such as in 2-S-pyridyl:

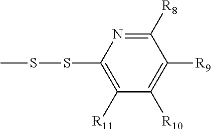

wherein, R₈₋₁₁ are independently selected from among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;
to react with a thiol in C1-inhibitor.

In a further and/or alternative embodiment, bifunctional linkers include an amino acid. The amino acid which can be selected from any of the known naturally-occurring L-amino acids is, e.g., alanine, valine, leucine, isoleucine, glycine, serine, threonine, methionine, cysteine, phenylalanine, tyrosine, tryptophan, aspartic acid, glutamic acid, lysine, arginine, histidine, proline, and/or a combination thereof, to name a few. In alternative aspects, L can be a peptide residue. The peptide can range in size, for instance, from about 2 to about 10 amino acid residues (e.g., 2, 3, 4, 5, or 6).

Derivatives and analogs of the naturally occurring amino acids, as well as various art-known non-naturally occurring amino acids (D or L form), hydrophobic or non-hydrophobic, are also contemplated to be within the scope of the invention. Simply by way of example, amino acid analogs and derivatives include:

2-aminoadipic acid, 3-aminoadipic acid, beta-alanine, beta-aminopropionic acid,

2-aminobutyric acid, 4-aminobutyric acid, piperidinic acid, 6-aminocaproic acid,

2-aminoheptanoic acid, 2-aminoisobutyric acid, 3-aminoisobutyric acid,

2-aminopimelic acid, 2,4-aminobutyric acid, desmosine, 2,2-diaminopimelic acid,

2,3-diaminopropionic acid, N-ethylglycine, N-ethylasparagine, 3-hydroxyproline,

4-hydroxyproline, isodesmosine, allo-isoleucine, N-methylglycine or sarcosine,

N-methylisoleucine, 6-N-methyllysine, N-methylvaline, norvaline, norleucine, ornithine, and others too numerous to mention, that listed in 63 Fed. Reg., 29620, 29622 are incorporated herein by reference.

One embodiment of the L groups includes glycine, alanine, methionine or sarcosine.

Additional linkers are found in Table 1 of Greenwald et al. (Bioorganic & Medicinal Chemistry, 1998, 6:551-562), and in U.S. Pat. Nos. 6,180,095, 6,720,306, 5,965,119, 6,303,569, 6,624,142, 7,122,189, 7,897,647, 7,087,229, and 7,413,738, the contents of each of which are incorporated by reference herein.

### Synthesis of Conjugates of Formula (I)

Several examples of synthesis of the polymeric conjugates of C1-inhibitor using polyethylene glycols (PEG) of the present invention are provided in the following schemes.

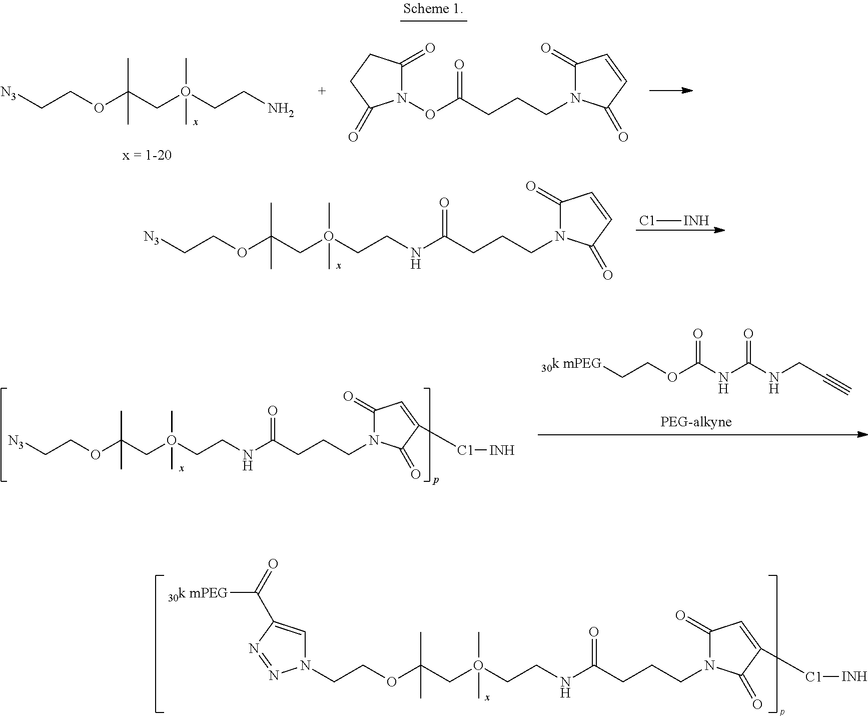

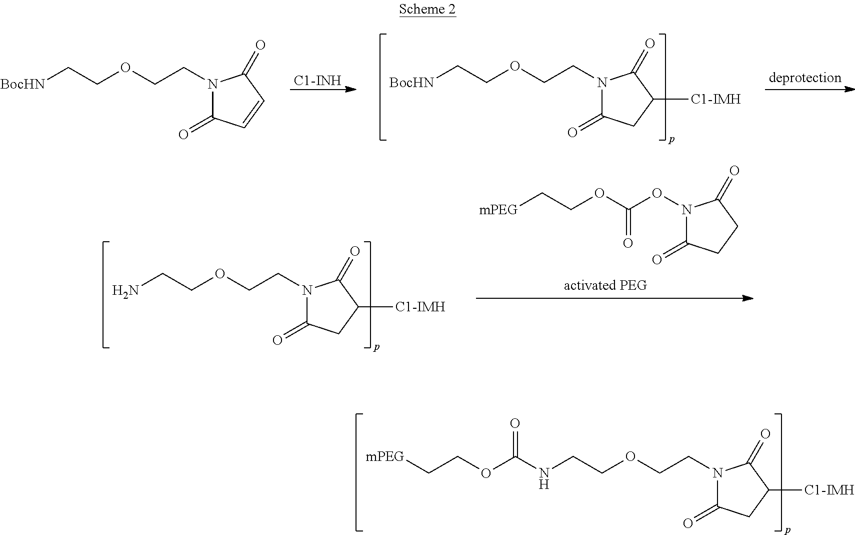

wherein p is 1 or an integer less than or equal to the number of available cyseine or thiol sites found on the C1-Inhibitor target.

Generally, the conjugates described herein are prepared by reacting a C1-inhibitor with a polyalkylene oxide having a thiol or specific functional group or an activating group, under aqueous conditions sufficient to form a covalent bond between the polyalkylene oxide and thiol group of an amino acid of the C1-esterase inhibitor and purifying the resulting conjugate.

In an alternative embodiment, the activating group is a vinyl group and the reaction is carried out in the presence of a reducing agent. A vinyl group or disulfide group is used for attachment of the polymer to a thiol on the C1-inhibitor. For certain thiols, there is structural hindrance in a large molecule such as a protein and the PEGs which limits the conjugation efficiency. The present invention provides a method to change or unfold the conformation of C1 inhibitor to expose the thiols located in a sterically hindered area or even assist in breaking a disulfide bond in the presence of a high salt concentration or a denaturing agent and/or a reducing agent for conjugation with a polymer. The sterically hindered thiols or thiols from disulfide are further reacted with a small bifunctional compound to further improve the conjugation with a polymer. The small bifunctional compound, or an “extension,” assists for the polymer to reach the sterically blocked thiols by providing a terminal functional moiety at the distal end of the bifunctional compound from the thiol and thus, providing more space for the polymer for conjugation.

One of the advantages of the present invention is to expose the sterically hidden or blocked disulfide bonds without a presence of a reducing agent but by conformational stress from the denaturation of the protein, C1 inhibitor. Elimination of any harsh reducing agents for hydrolysis of the disulfide bond can conserve chemical stability of the peptides bonds in the protein.

In other aspects of the invention, the other activated linkers known in the art will allow for non-specific linkage of the polymer to cysteine thiol groups-forming thio ether, thio carbamate (urethane) or thio amide linkages. Such activated linkers can be reacted in molar excess with the target C1-inhibitor under conditions well known to those of ordinary skill. The activating group in the linker or polymer can be selected from among carbonyl imidazole, chloroformate, isocyanate, PNP, tosylate, N-HOBT, and N-hydroxysuccinimidyl, for example, in order to react with the distal end of the bifunctional spacer from C1-inhibitor.

For purposes of illustration, suitable conjugation reactions include reacting C1-inhibitor with a suitably activated polymer system described herein. The reaction is preferably carried out using conditions well known to those of ordinary skill for protein modification, including the use of a PBS buffered system, etc. with the pH in the range of about 5.0-5.5. It is contemplated that in most instances, an excess of the activated polymer will be reacted with the C1-inhibitor.

Reactions of this sort will often result in the formation of conjugates containing one or more polymers attached to the C1-inhibitor. As will be appreciated, it will often be desirable to isolate the various fractions and to provide a more homogenous product. In most aspects of the invention, the reaction mixture is collected, loaded onto a suitable column resin and the desired fractions are sequentially eluted off with increasing levels of buffer. Fractions are analyzed by suitable analytical tools to determine the purity of the conjugated protein before being processed further.

It will also be appreciated that heterobifunctional polyalkylene oxides are also contemplated for purposes of cross-linking C1-inhibitor, or providing a means for attaching other moieties such as targeting agents for conveniently detecting or localizing the polymer-C1-inhibitor conjugate in a particular areas for assays, research or diagnostic purposes.

### Formulations

Polymer conjugates of the present invention may be manufactured and formulated by processes well known in the art, e.g., using a variety of well-known mixing, dissolving, granulating, levigating, emulsifying, encapsulating, entrapping or lyophilizing processes. Compositions may be formulated in conjunction with one or more physiologically acceptable carriers comprising excipients and auxiliaries which facilitate processing of the active conjugates into preparations which can be used pharmaceutically. Proper formulation is dependent upon the route of administration chosen. Parenteral routes are preferred in many aspects of the invention, but not limited to.

In another aspect, the conjugates may also be formulated for parenteral administration or injection, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. For injection, including, without limitation, intravenous, intramuscular and subcutaneous injection, the polymer conjugates of the invention may be formulated in aqueous solutions, preferably in physiologically compatible buffers such as physiological saline buffer or polar solvents including, without limitation, a pyrrolidone or dimethylsulfoxide. Aqueous injection suspensions may contain substances that increase the viscosity of the suspension, such as sodium carboxymethyl cellulose, sorbitol, or dextran. Additionally, suspensions of the active conjugates may be prepared in a lipophilic vehicle. Suitable lipophilic vehicles include fatty oils such as sesame oil, synthetic fatty acid esters such as ethyl oleate and triglycerides, or materials such as liposomes. Optionally, the suspension may also contain suitable stabilizers and/or agents that increase the solubility of the conjugates to allow for the preparation of highly concentrated solutions. Alternatively, the active ingredient may be in powder form, such as lyophilized product, for constitution with a suitable vehicle, e.g., sterile, pyrogen-free water, before use.

### Methods of Administration and Dosage

The C1-inhibitor polymer conjugate described herein is useful for all of the methods and indications already art-known for Cinryze® (Viro Pharma Biologics, Inc.) and Berinert® (CSL Behring LLC). Thus, the inventive C1-inhibitor conjugate is administered to a patient in need thereof in an amount that is effective to treat a disease or disorder or other condition that is responsive to such treatment. The artisan will appreciate suitable amounts, routes of administration and dosing schedules extrapolated from the known properties of Cinryze® and Berinert®.

Another aspect of the present invention provides methods of treatment for various medical conditions in mammals, preferably humans. The methods include administering an effective amount of a pharmaceutical composition that includes a C1-inhibitor polymer conjugate prepared as described herein, to a mammal in need of such treatment. The conjugates are useful for, among other things, treating C1-inhibitor-susceptible conditions or conditions which would respond positively or favorably as these terms are known in the medical arts to C1-inhibitor-based therapy.

Conditions that can be treated in accordance with the present invention are generally those that are susceptible to treatment with C1-inhibitor. Exemplary conditions which can be treated with C1-inhibitor include, but are not limited to, ongoing, acute attacks of hereditary angioedema (HAE) affecting the abdomen, face or throat in adults and adolescents and all other medical conditions know to those of ordinary skill to benefit from C1-inhibitor therapy. In a preferred aspect of the invention, the polymer conjugated C1-inhibitor is administered to patients in amounts effective to treat hereditary angioedema or prevent swelling and/or painful attacks in teenagers and adults with Hereditary Angioedema.

Administration of the described dosages may be every other day, but is preferably once or twice a week. Doses are usually administered over at least a 24 week period by injection or infusion. Administration of the dose can be intravenous, subcutaneous, intramuscular, or any other acceptable systemic method, including subdermal or transdermal injection via conventional medical syringe and/or via a pressure system. Based on the judgment of the attending clinician, the amount of drug administered and the treatment regimen used will, of course, be dependent on the age, sex and medical history of the patient being treated, the stage or severity of the specific disease condition and the tolerance of the patient to the treatment as evidenced by local toxicity and by systemic side-effects. Dosage amount and frequency may be determined during initial screenings of neutrophil count.

The amount of the C1-inhibitor polymer conjugate composition administered to treat the conditions described above is based on the C1-inhibitor activity of the polymeric conjugate. It is an amount that is sufficient to significantly affect a positive clinical response. Although the clinical dose will cause some level of side effects in some patients, the maximal dose for mammals including humans is the highest dose that does not cause unmanageable clinically-important side effects. For purposes of the present invention, such clinically important side effects are those which would require cessation of therapy due to severe flu-like symptoms, central nervous system depression, severe gastrointestinal disorders, alopecia, severe pruritus or rash. Substantial white and/or red blood cell and/or liver enzyme abnormalities or anemia-like conditions are also dose limiting.

A therapeutically effective amount refers to an amount of conjugate effective to prevent, alleviate or ameliorate the C1-inhibitor-susceptible condition. Determination of a therapeutically effective amount is well within the capability of those skilled in the art, especially in light of the disclosure herein.

The dosage, of course, can vary depending upon the dosage form and route of administration. The exact formulation, route of administration and dosage can be selected by the individual physician in view of the patient's condition.

For any conjugate used in the methods of the invention, the therapeutically effective amount may be estimated initially from in vitro assays. Then, the dosage can be formulated for use in animal models so as to achieve a circulating concentration range that includes the effective dosage. Such information can then be used to more accurately determine dosages useful in patients.

Toxicity and therapeutic efficacy of the conjugates described herein can be determined by standard pharmaceutical procedures in cell cultures or experimental animals using methods well-known in the art.

As explained above, the dosages of the polymer C1-inhibitor conjugate compositions of the present invention will vary somewhat depending upon the C1-inhibitor moiety and polymer selected. In general, however, the conjugate is administered in amounts ranging from about 100 to about 5,000 u/kg/week, from about 500 to about 4,000 u/kg/week or from about 1,000 to 3,000 u/kg/week of C1-inhibitor equivalent in the polymer conjugate, based on the condition of the treated mammal or human patient. The range set forth above is illustrative and those skilled in the art will determine the dosing of the conjugate selected based on clinical experience and the treatment indication.

The conjugates may be administered once daily or divided into multiple doses which can be given as part of a multi-week treatment protocol. The precise dose will depend on the stage and severity of the condition, the susceptibility of the condition to the C1-inhibitor polymer conjugate, and the individual characteristics of the patient being treated, as will be appreciated by one of ordinary skill in the art.

Practice of the invention would allow treatment of this condition, and others, at higher doses and in combination with other art-known therapeutic agents.

### EXAMPLES

The following examples serve to provide further appreciation of the invention but are not meant in any way to restrict the effective scope of the invention.

### Materials

Reagents: C1 Esterase Inhibitor was obtained from Athens Research & Technology and has MW: 73000 Da as determined by MALDI. Activated PEG's werer obtained from NOF;
Buffers: (1) 100 mM Na acetate, 150 mM naCl, pH5.5; (2) PBS
Ultrafiltration: 10 k Pellicon XL 50 Ultrafiltration Cassettes
Amicon Membrane: 30K Ultrafiltration Membrane (Millipore)
Sterile Filter: 0.2 μm sterile polyethersulfone filter (VWR)

### Purification of Mono and Di PEGylated C1 INH Conjugates

Mono or Di PEGylated C1-INH (both PEG linear and branched) was purified by weak anion exchange column (HiTrap DEAE FF, 1 ml. GE Healthcare) or by hydrophobic interaction column (HIC phenyl FF, 1 ml. GE Healthcare). In DEAE column purification, Buffer A contained 10 mM Tris, pH 8.5 and buffer B had 0.5 M NaCl in buffer A. Elution was conducted at 1 ml/min over 30 min. Based on SDS-PAGE, the majority components in flow through was di PEG-C1 INH. Mono PEG-C1 INH and native C1 INH were both bound to the column and started to elute out at ˜0.12 M NaCl. The fractions containing mono PEG-C1 INH identified by SDS-PAGE was concentrated using Centricon YM30 (Millipore) and the buffer was exchanged to PBS by NAP-5 column (GE Healthcare). In HIC phenyl purification, Buffer A contained 0.75 M ammonium sulfate in PBS buffer and buffer was PBS. Elution was conducted at 1 ml/min over 30 min. The first elution peak identified on SDS-PAGE was mono PEG-C1 INH and second peak was di PEG-C1 INH. Mono and di PEG-C1 INH were concentrated using Centricon YM30 and buffer-exchanged to PBS by NAP-5 column.

### Characterization of PEG-C1 INH

The concentration of PEGylated C1 INH was determined by UV at 280 nm. The Sample at 5 μg or 10 μg was loaded into the gel without sample reduction and heating for electrophoresis (Novex NuPAGE 10% Bis-Tris gel, Invitrogen). The protein bands were visualized after simple blue stain. The density of the image was obtained on Molecular Dynamics. As seen on SDS gel, all C1 INH was converted into PEGylated form.

### Example 1

### PEGylation of C1 Esterase Inhibitor with Maleimide mPEG

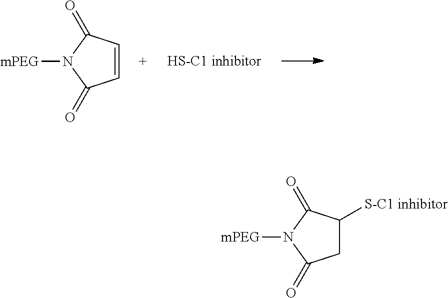

120 mg of native C1 inhibitor (12 vials×10 mg/vial of ARTlot C12012-02) was diluted to 110 ml by 100 mM Na phosphate, pH6.0. Added 12 ml of 100 mM DTT (freshly made in 100 mM Na phosphate, pH6.0) to the above native C1 inh and stirred at RT for 30 min. DTT was removed by a desalting column (BPG100 column packed with Sephadex G25 Medium, bed height 21.8 cm, pre-equilibrated with 100 mM Na phosphate, pH6.0) operated by Unicorn of AKTA (S/N: 01112957, Highflow, GE Healthcare, NJ). The reduced C1 inhibitor was collected in 270 ml. Was added 533 mg of 40 kDa maleimide-PEG to the above sample and stirred at RT for 2 hr. After PEGylation, the sample was loaded to another desalting column (BPG100 column packed with Sephadex G25 Medium, bed height 21.8 cm, pre-equilibrated with 20 mM Tris, pH8.0) operated by Unicorn of AKTA (S/N: 01112957, Highflow, GE Healthcare, NJ) and collected in a 500 ml bottle. The collected sample from the desalting column was loaded to a pre-equilibrated DEAE FF (XK26, 28 cm bed height) at 100 cm/h. The column was washed by 5 column volumes (CV) of the equilibration buffer and eluted in a 10 CV of linear gradient to 0.5 M NaCl in 20 mM Tris at pH8.0. The peak was fractionated in the 50 ml tubes and analyzed by a SDS-PAGE. The fractions collected from DEAE FF eluate were pooled, adjusted to 0.75 M ammonium sulfate in 20 mM Tris at pH8.0 and loaded to a HIC column (Phenyl HP, XK26, bed height 26 cm) pre-equilibrated by 0.75 M ammonium sulfate, 20 mM Tris, pH8.0 at 60 cm/h. The column was washed by 2 CV of equilibration buffer and eluted in 10 CV of linear gradient to the buffer of 20 mM Tris, pH8.0. The peak was fractionated in 50 ml tubes and analyzed by SDS-PAGE. The fractions collected from Phenyl HP column were pooled, concentrated in an Amcicon® 8050 installed with one piece of 10 K Ultrafiltration Membrane (Cat# PLGC0431, Lot# C1SA5784, Millipore) in a cold room to <3 ml, then added 100 ml of PBS (pH7.4) and concentrated to <3 ml again;

Repeated concentration/dilution process 5 times until the all the ammonium sulfate was removed. Pipetted the sample out and rinsed the membrane two 5 ml of PBS (pH7.4). Combined the sample with the rinse in a tube, resulting 18 ml. Filtered this sample by a 0.2 μm sterile polyethersulfone filter (North American Cat#28145-501, Batch#21294, VWR). The resultant conjugates were purified using standard chromatogram purification techniques.

Mal-PEG-C1 INH after Purification

The final Mal-PEG-C1 INH in vitro C1 inhibitory activity was determined to be 6.1 U/mg, which was 86% of native C1 inhibitor. There no free PEG or native C1 inhibitor found as evaluated by RP-HPLC.

### Example 2

### Preparation of Pegylated C1 Inhibitor with Branched 40k mPEG-Maleimide

100 mg of native C1 inhibitor (12 vials×10 mg/vial of ARTlot C12012-04) was diluted to 90 ml by 100 mM Na phosphate, pH6.0. Reduction: added 10 ml of 100 mM DTT (freshly made in 100 mM Na phosphate, pH6.0) to the above native C1 inh and stirred at RT for 30 min. DTT was removed by a desalting column (BPG100 column packed with Sephadex G25 Medium, bed height 21.8 cm, pre-equilibrated with 100 mM Na phosphate, pH6.0) operated by Unicorn of AKTA (S/N: 01112957, Highflow, GE Healthcare, NJ). The reduced C1 inhibitor was collected in 280 ml. 400 mg of 40 kDa maleimide U-PEG was added to the above sample and stirred at RT for 2 hr. After PEGylation, buffer was exchanged as the sample was loaded to another desalting column (BPG100 column packed with Sephadex G25 Medium, bed height 21.8 cm, pre-equilibarted with 20 mM Tris, pH8.0) operated by Unicorn of AKTA (S/N: 01112957, Highflow, GE Healthcare, NJ) and collected in a 500 ml bottle.

The above sample was loaded to a pre-equilibrated DEAE FF (XK26, 28 cm bed height) at 100 cm/h. The column was washed by 5 column volumes (CV) of the equilibration buffer and eluted in a 10 CV of linear gradient to 0.5 M NaCl in 20 mM Tris at pH8.0. The peak was fractionated in 50 ml tubes and analyzed by SDS-PAGE. The fractions collected from DEAE FF eluate was adjusted to 0.75 M ammonium sulfate in 20 mM Tris at pH8.0 and loaded to a HIC column (Phenyl HP, XK26, bed height 26 cm) pre-equilibrated by 0.75 M ammonium sulfate, 20 mm Tris, pH8.0 at 60 cm/h. The column was washed by 2 CV of equilibration buffer and eluted in 10 CV of linear gradient to the buffer of 20 mM Tris, pH8.0. The peak was fractionated in 50 ml tubes and analyzed by SDS-PAGE. The fractions collected from Phenyl HP column was concentrated in an Amcicon® 8050 installed with one piece of 10 K Ultrafiltration Membrane (Cat# PLGC0431, Lot# C1SA5784, Millipore) in a cold.

Repeated the concentration/dilution process 5 times until the all the ammonium sulfate was removed. Pipetted the sample out and rinsed the membrane by 5 ml of PBS. Filtered this sample by a 0.2 μm sterile polyethersulfone filter (North American Cat#28145-501, Batch#21294, VWR). The conjugates were purified as mentioned above using standard chromatogram purification techniques. The final Mal-U-PEG-C1 INH had in vitro C1 inhibitory activity of 3.3 U/mg, which was 47% of native C1 inhibitor. There no free PEG or native C1 inhibitor found In Mal-U-PEG-C1 INH as evaluated by RP-HPLC.

### Example 3

### PEGylation of C1 Esterase Inhibitor Via Disulfide Bond without Protein Reduction

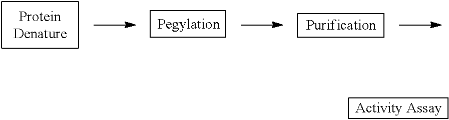

C1 inhibitor was prepared at 1 mg/ml in the denaturing buffer (1) 3 M guandine-HCl, 3 M Urea, 100 mM Na phosphate, 2 mM EDTA, pH6.0, and (2) 3 M guandine-HCl, 3 M Urea, 100 mM Na phosphate, 2 mM EDTA, pH6.0. Protein denaturing took place at room temperature for 30 min before pegylation started. Pegylation of C1 inhibitor with maleimide mPEG (linear or branched) or PEG-SS-2-pyridyl was performed in the buffer of 100 mM Na phosphate, 2 mM EDTA, pH6.0 with the mole ratio of protein:PEG at ˜10:1. The pegylation was quenched by 3 mM freshly prepared cysteine after PEGylation at room temperature 3 h. Under these conditions, monoPEG-C1 inhibitor can be achieved at about 25% of total protein. The pegylated C1 inhibitor was adjusted to 0.75 M ammonium sulfate and loaded to an appropriate size of hydrophobic interaction chromatography column (HIC), e.g., Phenyl HP (GE Healthcare, NJ) pre-equilibrated with the equilibration buffer, which contains 0.75 M Ammonium sulfate, 20 mM Na phosphate, pH7.0. The column was washed to baseline with the equilibration buffer for 5 extra column volumes before the linear gradient elution against the buffer of 20 mM Na phosphate pH7.0. pegylated C1 inhibitor was collected in fractions and buffer exchanged to PBS before submission for activity assay and characterizations.

### Example 4

### PEGylation of C1 Esterase Inhibitor Via Disulfide Bond with Protein Reduction

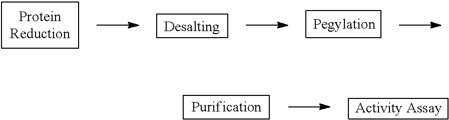

C1 inhibitor (1 mg/ml) was reduced bt DTT at 10 mM at room temperature for 10 min in the buffer of 100 mM Na phosphate, 2 mM EDTA, pH6.0. The reduced C1 inhibitor was desalted by a desalting column (eg., PD-10, ANP5, or any desalting columns made GE Healthcare, Thermo Scientific, Bio Rad, etc. Pegylaion of c1 inhibitor with maleimide mPEG (linear or branched) was performed in the buffer of 100 mM Na phosphate, 2 mM EDTA, pH6.0 with the mole ratio of protein:PEG at ˜10:1. The pegylation was quenched by 3 mM freshly prepared cysteine after pegylation at room temperature for 3 hours. Under these conditions, monoPEG-C1 inhibitor can be achieved at about >95%% of total protein. The pegylated C1 inhibitor was adjusted to 0.75 M ammonium sulfate and loaded to an appropriate size of hydrophobic interaction chromatography column (HIC), e.g., Phenyl HP (GE Healthcare, NJ) pre-equilibrated with the equilibration buffer, which contains 0.75 M Ammonium sulfate, 20 mM Na phosphate, pH7.0. The column was washed to baseline with the equilibration buffer for 5 extra column volumes before the linear gradient elution against the buffer of 20 mM Na phosphate pH7.0. pegylated C1 inhibitor was collected in fractions and buffer exchanged to PBS before submission for activity assay and characterizations.

### Example 5

### PEGylation of C1 Inhibitor Via Thiols from Disulfide Bond

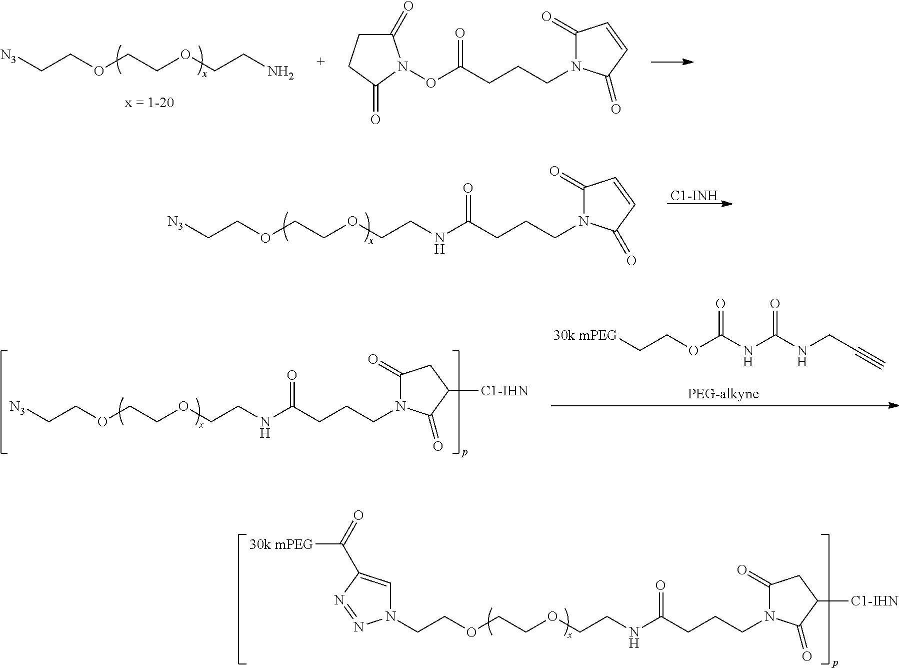

wherein p is as defined above.

### Linker Preparation:

N-Succinimidyl 4-Maleimidobutyrate and Azido-dPEG₁₁-amine were mixed in DMSO at the molecular ratio of 1:1.5 (e.g., 23 mg of N-Succinimidyl 4-Maleimidobutyrate+70 mg of Azido-dPEG₁₁-amine (MW570.67), mixed in 500 μl of DMSO) and stirred at RT for 30 min.

Linker Conjugation with Squeezed C1 INH

To a solution of C1-inhibitor of 460 μl (10 mg/ml), 2 ml of 4 M Guanidine-HCl, 4 M Urea, 100 mM Na phosphate, 2 mM EDTA, pH6.0 (final 3 M Urea, 3 M guandine-HCl), was added 40 μl of linker, vertexed, and stirred at RT for 3 hours.

Linker Conjugation with Reduced C1 INH
Reduction of C1 inh: 460 μl (10 mg/ml) was added 40 μl of 100 mM DTT, vertexed, and stirred at RT for 30 min. Then the reduced C1 inh was desalted by a MiniPD G-25 (GE Healthcare, NJ) column pre-equilibrated in 100 mM Na phosphate, 2 mM EDTA, pH6.0, resulting 1 ml
Added 1.5 ml of 100 mM Na phosphate, 2 mM EDTA, pH6.0, and 40 μl of linker, vertexed, and stirred at RT for 3 h

### Conjugation of Polymer Via Click Reaction

Desalting: 2.5 ml of C1 inh connected with linkers was desalted by PD-10 column pre-equilibrated in PBS, resulting 3.5 mg/ml
Click with alkyne-PEG: 0.5 ml of desalted C1 inh-linker was added PEG-alkyne (30K) at mole ratio of 1:100, and clicked at the presence of 1.18 mM Cu(I) and 1.15 mM of ligand (tris-Benzyltriazolymethy amine, TBTA) at RT overnight.

### Results

Yield comparison between Direct PEGylation by PEG-ss-NPYS and Extended bifunctional linker PEGylation evaluated by SEC-HPLC provides that higher yield of PEGylation was obtained by using denaturation approach or by employing the bifunctional spacer. The specific and enzyme activities of the PEGylated product using the bifunctional spacers or by denaturation approach were comparable to confirm that the protein's biological activity was not affected by the denaturation conditions or by the presence of the bifunctional spacers.

### Example 6

### PEGylation of C1 Inhibitor Via Thiols from Disulfide Bond

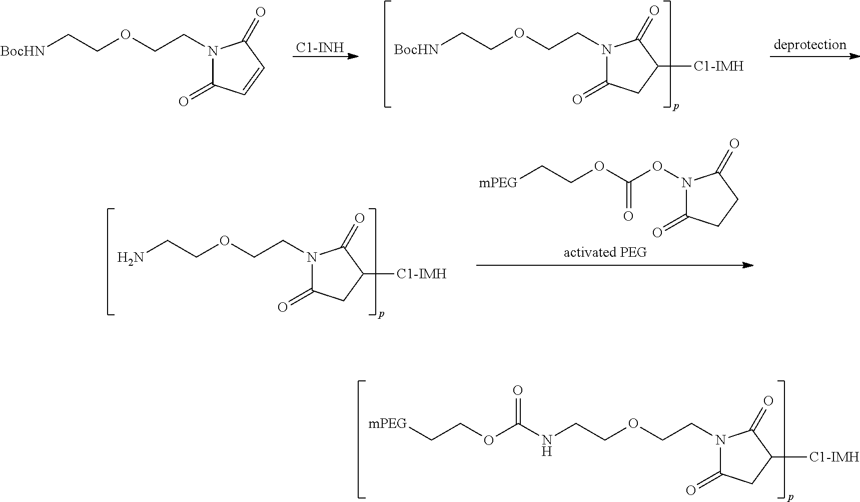

wherein p is as defined above.

### Example 7

### C1 Inhibitor Activity Assay

The inhibitory activity of C1 inhibitor against C1 esterase was evaluated the C1 activity after mixed with the inhibitor using a standard assay kit, TechnoChrom® C1-inh (30T), ref#5345003, TechoClone, Australia. Briefly, samples, standards, and controls were diluted in Tris-NaCl Buffer, then transferred to a 96-well plate, C1-Esterase was added to each well and the plate was incubated for 10 min 37° C. Then added substrate to each well and read plate at 37° C. for 4 minutes kinetically, plotted Response (DOD/min vs Standard Concentrations), Interpolated C1-INH concentration from Standard Curve.

The C1 esterase inhibitor protein has to bind to another enzyme to have activity. Thus, indiscriminate chemical modification could result in complete loss or significant reduction of biological activity.

The polymer conjugates of the present invention retained significant amount of C1-esterase inhibitor activity. It was a surprising result because it was speculated that modification of the active domain, C-terminal, can reduce the activity dramatically. Without being bound to any theory, it is possible that the present PEG attached to the thiol was still flexible enough to provide freedom for C-terminal for the high inhibitory activity. The above results provide that PEGylation of the present invention did not alter the C1-esterase activity even after multiple PEGylation.

### Example 8

### In Vivo Pharmacokinetics

The polymeric conjugates of C1 inhibitor prepared are administered (i.v.) to groups of rat for in vivo plasma pharmacokinetic (PK) study at dose of 70 U/kg. The polymer conjugates of the invention such as ALD-PEG-C1 INH demonstrates improved half-lives compared to the native C1-esterase inhibitor. Some polymer conjugates had extended half-life of up to about 80 hours, with more than a 10 fold improvement over the native C1 inhibitor. This profile can provide a long lasting treatment regime such as once a week.

1. A polymer conjugate, comprising:
a C1-inhibitor having at least one substantially non-antigenic polymer covalently attached thereto via thiol group of the C1-inhibitor.

2. The polymer conjugate of claim 1, wherein the substantially non-antigenic polymer is a polyalkylene oxide.

3. The polymer conjugate of claim 2, wherein the polyalkylene oxide is PEG

4. The polymer conjugate of claim 1, wherein the C1-inhibitor is a human C1 esterase inhibitor (C1-INH).

5. The polymer conjugate of claim 1, wherein the C1-inhibitor is a polypeptide represented by SEQ ID NO:1 or SEQ ID NO: 2.

6. The polymer conjugate of claim 1, wherein one of the substantially non-antigenic polymer is attached to a cysteine, a thiol group generated from a disulfide bond of a cysteine, a free thiol in a cysteine and to a thiol from a disulfide bond of the C1 inhibitor.

7-9. (canceled)

10. The polymer conjugate of claim 1, wherein the polymer conjugate retains about 40-80% of the biological activity of the C1-inhibitor in its native form.

11. (canceled)

12. The polymer conjugate of claim 1, wherein the molecular weight of the substantially non-antigenic polymer ranges from about 2,000 to about 100,000 daltons.

13. The polymer conjugate of claim 1, wherein the substantially non-antigenic polymer is conjugated via thioether, thioamide bond or thiocarbamate bond.

14. The polymer conjugate of claim 3, wherein the conjugate comprises Formula (I) or (I′):
[PEG-(CH₂)ₙ-(L)ₘ]ₚ—(X)_{p′}—C1-inhibitor (I) or
[PEG-(CH₂)ₙ-(L)ₘ]ₚ—(X)ₚ—C1-inhibitor-(X′)_{q′}-[(L′)_{m′}-(CH₂)_{n′}-PEG]_{q′} (I′)
wherein
PEG is a linear, branched or multi-arm poly(ethylene glycol) having a terminal group —(CH₂CH₂O)—;
L or L′ is independently a linker or functional group suitable to react with thiol;
(m) or (m′) is independently 0 or 1;
(n) or (n′) is independently zero or a positive integer;
(p) or (q) is independently a positive integer; and
X or X′ is S, a thiol group of an amino acid or a thiol group generated from a disulfide bond in C1-inhibitor attached to the polymer; (p′) or (q′) is independently a positive integer same as (p) or (q), respectively, provided that (m), (m′), (n) and (n′) are not zero simultaneously.

15. (canceled)

16. The polymer conjugate of claim 14, wherein L is selected from the group consisting of:

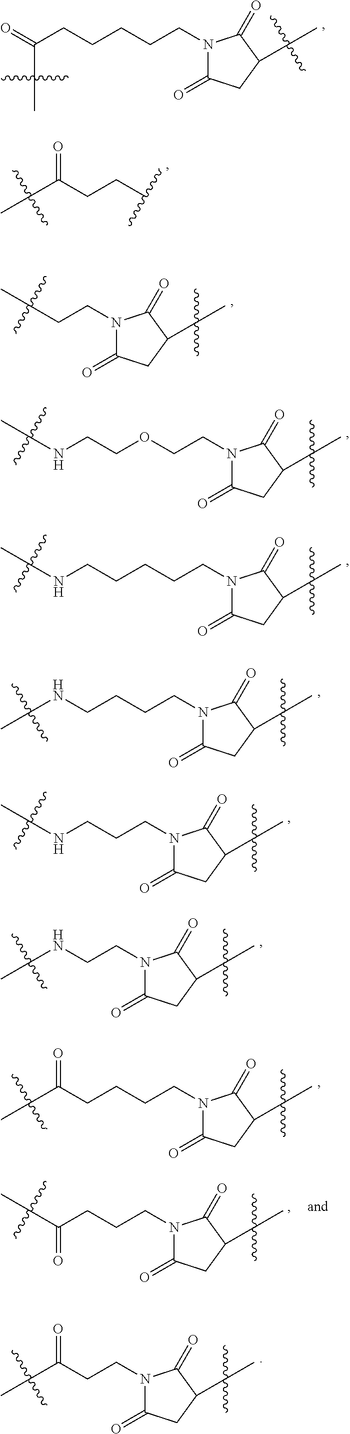

17. The polymer conjugate of claim 3 selected from the group consisting of: 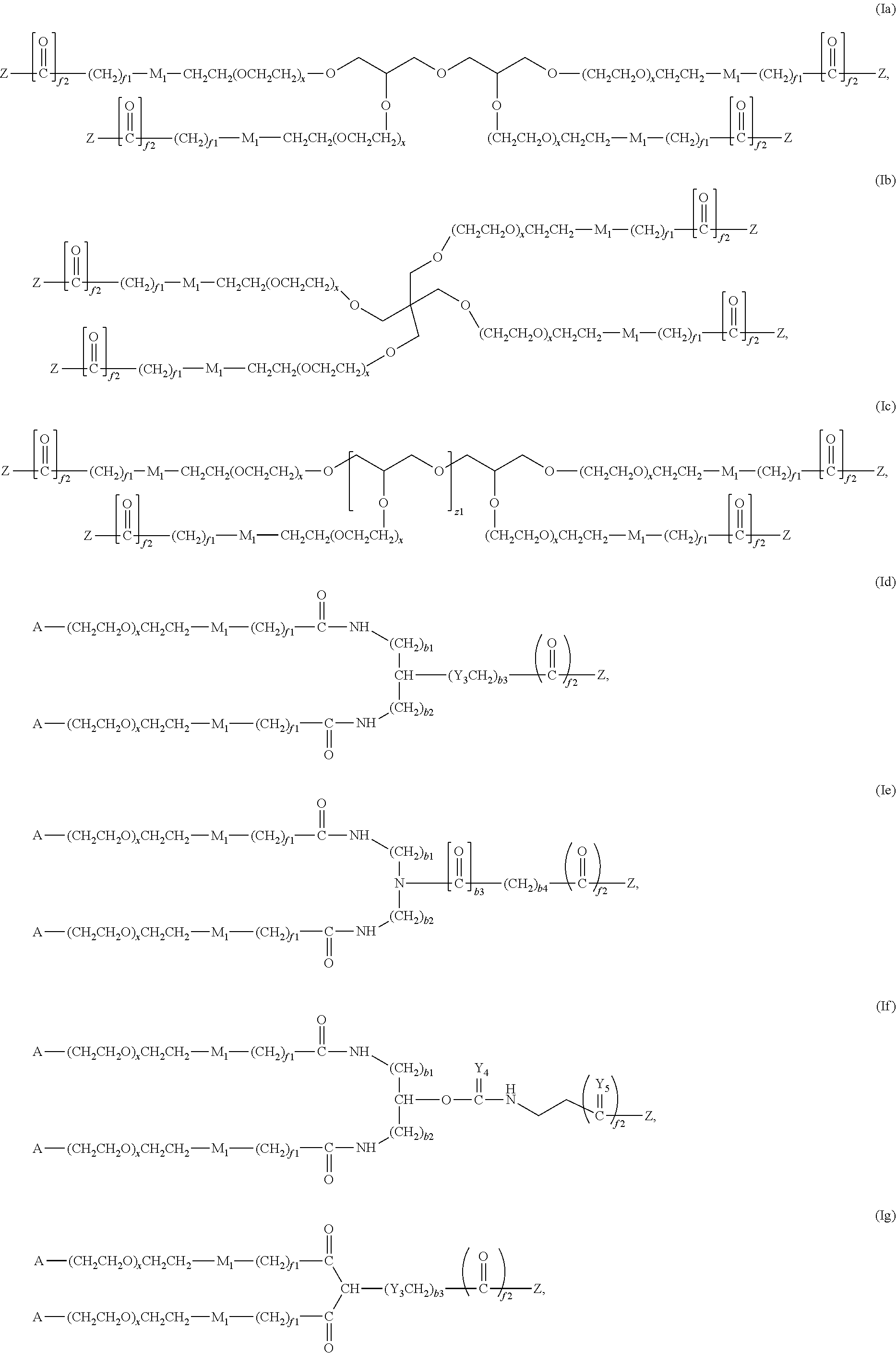
Z—[C(═O)]_{f2}—(CH₂)_{f1}-M₁-CH₂CH₂—O—(CH₂CH₂O)ₓ—CH₂CH₂-M₁-(CH₂)_{f1}-[C(═O)]_{f2}—Z, and (Ih)
A-(CH₂CH₂O)ₓ—CH₂CH₂-M₁-(CH₂)_{f1}-[C(═O)]_{f2}—Z, (Ii)
wherein
A is hydroxyl, NH₂, CO₂H, or C₁₋₆ alkoxy;
M₁ is O, S, or NH;
Y₃ is O, NR₅₁, S, SO or SO₂;
Y₄ and Y₅ are independently O, S or NR₅₁;
R₅₁, in each occurrence, is independently hydrogen, C₁₋₈ alkyl, C₁₋₈ branched alkyl, C₁₋₈ substituted alkyl, aryl, or aralkyl;
Z, in each occurrence, is independently OH, a leaving group, a targeting group, C₁₋₈ alkyl, C₁₋₈ alkoxy or C1 inhibitor containing moiety;
(b1) and (b2) are independently zero or positive integers;
(b3) is zero or 1;
(b4) is a positive integer;
(f1) is zero or a positive integer of from about 1 to about 10;
(f2) is zero or 1;
(z1) is zero or a positive integer of from 1 to about 27;
(x) is a degree of polymerization positive integer of from about 10 to about 2,300 so that the polymeric portion of the compound has the total number average molecular weight of from about 2,000 to about 100,000 daltons, provided that one or more Z are C1 inhibitor containing moiety.

18. The polymer conjugate of claim 3 selected from the group consisting of: 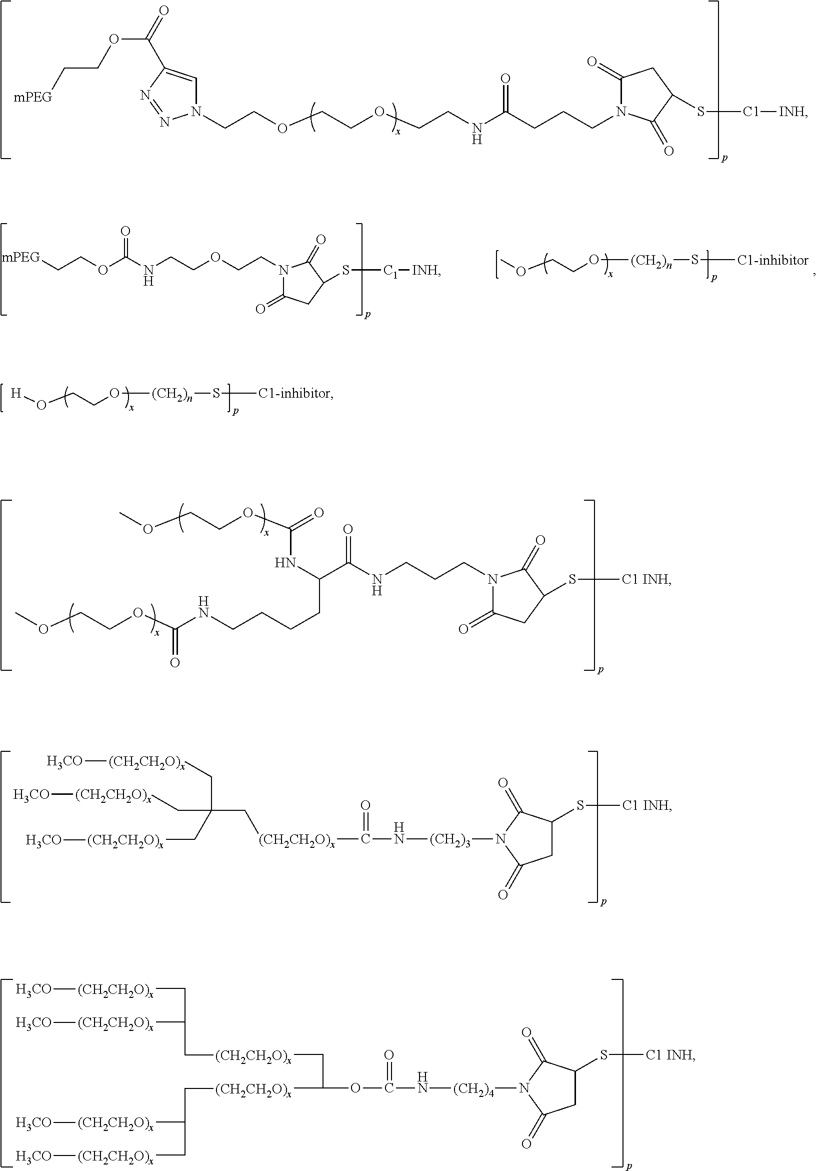 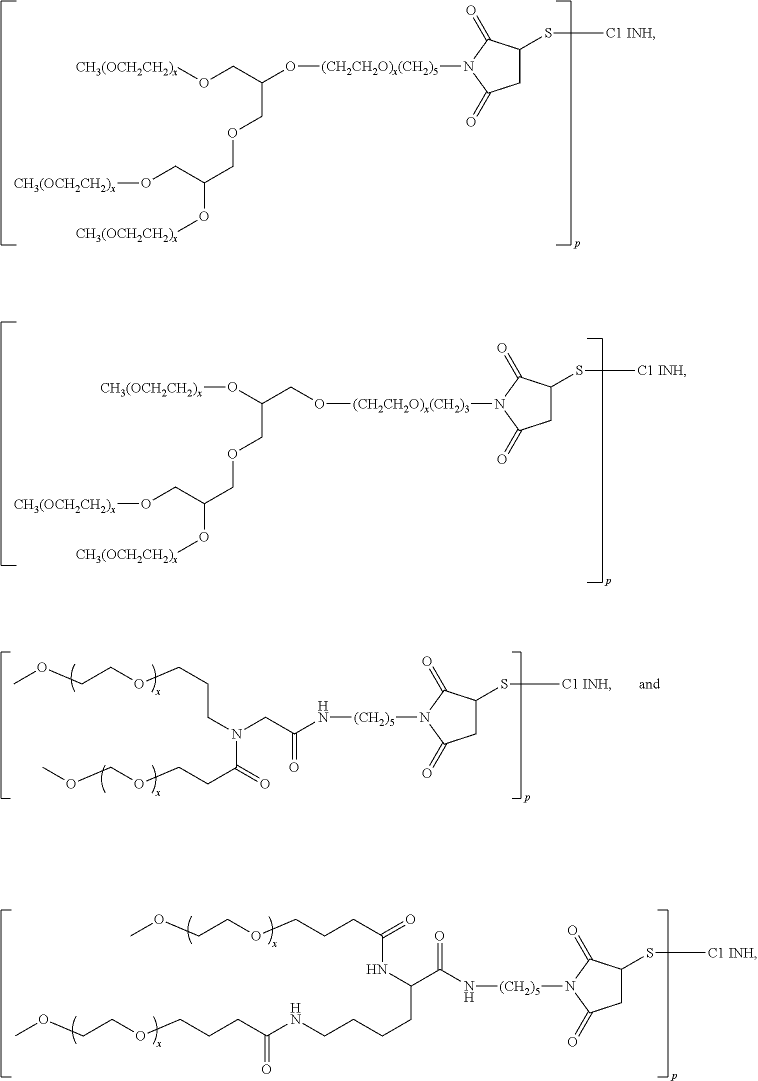
wherein,
(x) is a degree of polymerization positive integer of from about 10 to about 2,300 so that the polymeric portion of the compound has the total number average molecular weight of from about 2,000 to about 100,000 daltons; and
(p) is a positive integer.

19. A method of preparing a polymer conjugate comprising a C1-esterase inhibitor having at least one polyalkene oxide attached thereto via a thiol group of the C1-inhibitor, the method comprising:
reacting C1-esterase inhibitor with a polyalkylene oxide having an activating group, under conditions sufficient to form a covalent bond between the polyalkylene oxide and thiol group of an amino acid of the C1-esterase inhibitor; and
purifying the resulting conjugate.

20. The method of claim 19, wherein the activating group is selected from the group consisting of vinyl, sulfone, maleimide, and S-Pyridyl.

21. The method of claim 19, wherein the activating group is a maleimide and the reaction is carried out in the presence of a reducing agent.

22. A method of treating a mammal comprising administering an effective amount of a polymer conjugate of claim 1 to a patient in need thereof.

23. The method of claim 21, wherein the polymer conjugate is administered in amounts from about 100 u/kg/week to about 5,000 u/kg/week of C1-inhibitor equivalent in the polymer conjugate.

24. The method of claim 21, wherein the polymer conjugate is administered in amounts from about 500 u/kg/week to about 4000 u/kg/week of C1-inhibitor equivalent in the polymer conjugate.


SEQUENCE LISTING (2 sequences)

SEQ ID NO: 1 - protein, 478 aa, Homo sapiens
NPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 2 - protein, 500 aa, Homo sapiens
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA